(19) United States
(12) Patent Application Publication
(10) Pub. No.: US 2026/0219253 A1
(43) Pub. Date: Jul. 30, 2026

(54) METHOD FOR INTELLIGENT QUALITY EVALUATION OF INSTANT GREEN TEA BASED ON MULTI-SOURCE DATA FUSION AND USE THEREOF

(71) Applicants: Shanghai Jiao Tong University, Shanghai (CN); Hangzhou Mingbao Biotechnology Co., Ltd, Hangzhou City (CN); Shanghai Normal University, Shanghai (CN)

(72) Inventors: Xinlin WEI, Shanghai (CN); Yang WEI, Shanghai (CN); Wuxia YE, Hangzhou City (CN); Qun YE, Hangzhou City (CN); Wuji YE, Hangzhou City (CN); Yuanfeng WANG, Shanghai (CN); Chao KANG, Shanghai (CN); Kang WEI, Shanghai (CN); Lanlan PENG, Shanghai (CN)

(21) Appl. No.: 19/453,487

(22) Filed: Jan. 20, 2026

(30) Foreign Application Priority Data
Jan. 24, 2025 (CN) 202510118854.8

(51) Int. Cl. G01N 33/14; G06V 10/56; G06V 10/70; G06V 10/766; G06V 10/77; G06V 10/771; G06V 10/776; G06V 20/68; G16C 20/20

(52) U.S. Cl. CPC G01N 33/14; G06V 10/56; G06V 10/766; G06V 10/771; G06V 10/7715; G06V 10/776; G06V 10/87; G06V 20/68; G16C 20/20

(57) ABSTRACT

Provided are a method for intelligent quality evaluation of instant green tea based on multi-source data fusion and use thereof, belonging to the field of food inspection and involving the establishment of a system for intelligent quality identification of instant green tea. A rapid intelligent tea quality evaluation technique is mainly provided. Quality indicators of instant green tea are determined by chemical analysis; relevant data is collected using an intelligent evaluation technique; and correlation analysis is employed to reveal the relationships between the quality indicators and intelligent features. Based on independent data sources, a classification model for instant green tea varieties is constructed. By combining the multi-source data fusion technology with machine learning algorithms in this application, a system and method for efficient, accurate, and comprehensive intelligent quality evaluation of instant green tea are established.

### CROSS REFERENCE TO RELATED APPLICATION

This patent application claims the benefit of Chinese Patent Application No. 202510118854.8, filed Jan. 24, 2025, the disclosure of which is incorporated by reference herein in its entirety.

### TECHNICAL FIELD

The present disclosure relates to the technical field of food detection, and in particular, to a method for intelligent quality evaluation of instant green tea based on multi-source data fusion and use thereof.

### BACKGROUND

As a convenient and fast beverage, instant green tea is widely popular due to its unique flavor and health benefits. As the modern lifestyles become faster-paced, consumer demand for the quality of instant tea is continuously increasing. However, the quality variations among different varieties of instant green tea significantly affect consumer choices and brand competitiveness in the market. Currently, traditional quality evaluation methods typically rely on manual sensory evaluation and chemical analysis, but their subjectivity and inconsistency limit the accuracy and reliability of evaluations. Therefore, there is an urgent need to explore more objective and efficient intelligent evaluation techniques to improve the accuracy and consistency of quality inspection for instant green tea. With the rapid development of the intelligent evaluation techniques, it has become possible to assess the quality of instant green tea using computer vision, electronic noses, and electronic tongues. Moreover, the content of key compounds in instant green tea, such as tea polyphenols, catechins, and caffeine, directly affects the quality and nutritional value of the instant green tea. Therefore, research into rapid quantitative analysis methods for these key components holds both theoretical significance and practical application value.

In recent years, the rapid development of the intelligent evaluation techniques has provided new perspectives and methods for tea quality evaluation. These techniques, including advanced sensor technologies such as computer vision, electronic noses, and electronic tongues, enable efficient, accurate, and comprehensive analysis of the appearance, aroma, and taste of tea.

Currently, research on the application of intelligent evaluation techniques in quality inspection for instant green tea remains relatively scarce both domestically and internationally. Existing studies predominantly focus on quality inspection aspects such as tea variety differentiation, grade classification, geographical origin identification, and processing monitoring.

Despite the lack of systematic data in application research on instant green tea, the introduction of intelligent evaluation techniques combined with machine learning algorithms holds promise for achieving more precise and rapid quality detection and analysis of instant green tea.

### SUMMARY

An objective of the present disclosure is to overcome the defects in the prior art as described above and to provide a method for intelligent quality evaluation of instant green tea based on multi-source data fusion and use thereof. By adopting advanced technical means, efficient, accurate, and comprehensive quality evaluation of instant green tea is achieved, thereby overcoming limitations associated with traditional quality evaluation methods, such as high cost, complex operation, and results being susceptible to subjective influences.

The present disclosure can be achieved by the following technical solutions.

A first objective of the present disclosure is to provide a method for intelligent quality evaluation of instant green tea based on multi-source data fusion, including:
step 1, selecting instant green tea as a raw material; brewing one portion of the instant green tea with boiling water to form a tea infusion, while keeping the other portion of the instant green tea as a tea powder; using a computer vision device to collect visual images of the tea infusion and the tea powder; using an electronic nose to acquire aroma information of the tea infusion and the tea powder; using an electronic tongue to collect taste information of the tea infusion; and applying high-performance liquid chromatography to measure information of quality components in the tea infusion and the tea powder;
step 2, performing dimensionality reduction separately on the aroma information, the visual images, and the taste information that are used as data of feature parameters; after the dimensionality reduction, dividing a dataset; determining quality indicators of the tea infusion and the tea powder; and performing correlation analysis and significance analysis on the quality indicators and the feature parameters to form a fused feature set;
step 3, selecting features from the fused feature set formed in step 2 to form an optimal feature subset; constructing, based on the optimal feature subset, regression and classification models of support vector machine (SVM), K-nearest neighbor (KNN), and random forest (RF), and prediction models of partial least squares regression (PLSR), support vector regression (SVR), and random forest regression (RFR) to predict contents of the quality indicators of the instant green tea, and using indicators of classification accuracy rate, R² value of training and prediction sets, root mean square error of calibration (RMSEC), root mean square error of prediction (RMSEP), and relative prediction deviation (RPD) as evaluation criteria; and
step 4, applying data fusion at a decision level and a feature level based on the optimal feature subset formed in step 3; applying a multiple linear regression (MLR) model and Dempster-Shafer (D-S) evidence theory in a model architecture at the decision level and introducing a feature selection method at the feature level to perform multi-source fusion on the data separately; deriving fusion models based on evaluation results to improve accuracy and stability of classification and prediction; for variety identification of the instant green tea, applying the model architecture at the decision level; for quality identification of the instant green tea, applying the model architecture at the feature level; and using the R² value, the RMSEC, the RMSEP, and the RPD as evaluation indicators for evaluation so as to select an optimal fusion model.

Further, the method for intelligent quality evaluation includes:
step 1, selecting instant green tea as a raw material, and brewing one portion of the instant green tea with boiling water at a certain ratio to form a tea infusion, while keeping the other portion of the instant green tea as a tea powder; using an image acquisition device to collect visual images; using an olfactory sensing detection system to acquire aroma information of the tea infusion and the tea powder; and using a gustatory sensing system to collect taste information of the tea infusion;
step 2, performing dimensionality reduction on olfactory, visual, and taste information as feature data; after the dimensionality reduction, dividing a dataset; extracting feature information from the data, performing correlation analysis, and then constructing a model training set for classification and prediction;
step 3, performing dimensionality reduction with principal component analysis (PCA) on feature parameters collected by a computer vision device, an electronic nose, and an electronic tongue in step 1 to reduce data redundancy and noise; measuring, by high-performance liquid chromatography, contents of 11 components related to matcha quality, including catechin monomers (epigallocatechin (EGC), catechin (C), epicatechin (EC), epigallocatechin gallate (EGCG), and epigallocatechin (EGC)) in 10 different varieties of instant green tea, as well as tea polyphenols (TPs), total free amino acids (FAAs), caffeine (CAF), and total catechins (TCs); and performing correlation analysis on quality indicators and the feature parameters to form a fused feature set;
step 4, selecting features from the fused feature set formed in step 3 to form an optimal feature subset; constructing, based on the optimal feature subset, regression and classification models of SVM, KNN, and RF, and prediction models of PLSR, SVR, and RFR to predict contents of the quality indicators of the instant green tea; and using indicators such as classification accuracy rate, R² value of training and prediction sets, RMSEC, RMSEP, and RPD as evaluation criteria; and
step 5, applying data fusion at a decision level and a feature level for variety classification and quality classification of the instant green tea; applying MLR and D-S evidence theory in a model architecture at the decision level; introducing four feature selection methods, i.e., Pearson score, recursive feature elimination (RFE), particle swarm optimization (PSO), and Lasso regression, at the feature level to perform multi-source fusion on the data; optimizing the models according to a plurality of evaluation results to improve the accuracy and stability of classification and prediction; for variety identification of the instant green tea, applying the model architecture at the decision level; for quality identification of the instant green tea, applying the model architecture at the feature level; and using the R² value, the RMSEC, the RMSEP, and the RPD as evaluation indicators.

Further, the computer vision device includes:
a dark chamber, a bracket, a light source, a camera, and a holding container;
the bracket, the light source, the camera, and the holding container are disposed inside the dark chamber;
the light source and the camera are connected to the bracket;
the camera, the light source, and the holding container are arranged sequentially from top to bottom; and
the holding container is configured to hold the tea infusion or the tea powder.

Further, the dark chamber includes a black matte acrylic plate and is in a cubic shape (400 mm×400 mm×600 mm); and a side sliding groove and a bottom groove are formed in a front side surface of a body of the dark chamber to allow a front panel to slide up and down and be inserted into the bottom groove, thereby forming an enclosed space inside the dark chamber to inhibit an external light environment from affecting sample collection;
the bracket includes a carrying platform (300 mm×300 mm), a support rod (400 mm high), and an adjusting bracket; the carrying platform is connected to the support rod, and the adjusting bracket is connected to the support rod; a stepped circular groove is designed at a central point of the carrying platform to secure the holding container to ensure a consistent position for each collection; and the adjusting bracket is configured to adjust distances from the light source and the camera to the container;
the light source is a D65 ring-shaped shadowless light source, as specified by the International Commission on Illumination (CIE), which approximates the true resolution of daylight and has a color temperature of 6500 K, an inner diameter of 75 mm, and an outer diameter of 120 mm; the light source can emit uniform and consistent light, thereby enabling the acquisition of higher-quality digital image information;
the camera, selected as Nikon D5100 (CMOS), is connected to a computer via a data cable, and parameters of the camera are adjusted using software to achieve image acquisition.

Further, the olfactory sensing detection system includes the electronic nose.

Further, the gustatory sensing system includes the electronic tongue.

Further, the regression and classification models (identification models) include one or more of SVM, KNN, and RF; and
the prediction model includes one or more of PLSR, SVR, and RFR.

Further, model evaluation indicators in the evaluation criteria and calculation methods thereof are as follows:

$$classification accuracy rate A = \frac{{N}_{c}}{N} ;$$

$$determination coefficient {R}^{2} value {R}^{2} = \frac{( {\sum }_{i = 1}^{n} {X}_{i} - \overline{X} ) {( {Y}_{i} - \overline{Y} )}^{2}}{{\sum }_{i = 1}^{n} ( {X}_{i} - \overline{X} ) {\sum }_{i = 1}^{n} ( {Y}_{i} - \overline{Y} )} ;$$

$$root mean square error of calibration R M S E C = \sqrt{\frac{{\sum }_{i = 1}^{n} {( {X}_{ci} - {Y}_{ci} )}^{2}}{n}} ;$$

$$root mean square error of prediction R M S E P = \sqrt{\frac{{\sum }_{i = 1}^{n} {( {X}_{pi} - {Y}_{pi} )}^{2}}{n}} ;$$

$$standard deviation S D = \sqrt{\frac{{\sum }_{i = 1}^{n} {( {X}_{i} - \overline{X} )}^{2}}{n - 1}} ; and$$

$$relative percentage deviation R P D = \frac{S D}{R M S E P}$$

where n represents a number of samples in the dataset; Xᵢ represents an actual value of an i-th sample during the prediction model construction; X̅ represents a mean of actual values of all samples during the prediction model construction; Yᵢ represents a predicted value of the i-th sample during the prediction model construction; and Y̅ represents a mean of predicted values of all samples during the prediction model construction.

Further, the established model is considered more reliable when the R² and the RPD are larger, and the RMSEC and the RMSEP are smaller. When the RPD is greater than or equal to 3.0, the predictive performance of the model is significant and satisfactory. When the RPD is between 2.5 and 3.0, the prediction of the model is acceptable but still has room for improvement. When RPD is less than or equal to 2.5, the predictive performance of the model is below an acceptable level and requires further optimization.

Further, the determination of the quality indicators of the tea infusion and the tea powder includes the following process:
determining the components in tea such as TPs, FAAs, the TP/FAA ratio, CAF, EGC, C, EC, and EGCG by liquid chromatography; for the tea powder, grinding a tea sample into powder (i.e., the tea powder), homogenizing the tea powder, then subjecting the tea powder to ultrasonic extraction with ultrapure water or a specific solvent, and centrifuging the extract; and filtering the supernatant for subsequent detection. A C-18 reversed-phase chromatography column is selected, and the contents of TPs, FAAs, CAF, and C, etc., are measured according to national standards, with the corresponding data recorded.

Further, in step 2, the dimensionality reduction is dimensionality reduction with PCA.

Further, the feature selection method includes one or more of four feature selection methods: Pearson score, recursive feature elimination (RFE), particle swarm optimization (PSO), and Lasso regression.

Further, Pearson correlation analysis is performed on 9 color feature parameters and the quality indicators of the instant green tea infusion and the tea powder. Meanwhile, the electronic nose technology is employed to extract 14 aroma feature parameters from the tea infusion and the tea powder, followed by an in-depth analysis of the correlations between these aroma features and 10 quality indicators. Additionally, correlation analysis is also performed on 18 taste feature parameters extracted by an electronic tongue sensor and the 10 quality indicators of the instant green tea. The TP content is measured according to GB/T 8313-2018 “Determination of total polyphenols and catechins content in tea”. The contents of catechins and CAF are measured according to GB/T 8313-2018 “Determination of total polyphenols and catechins content in tea”. The FAA content is measured according to GB/T 8314-2013 “Determination of free amino acids content in tea”.

Further, in the data fusion at the decision level, the MLR model is employed to classify different varieties of instant green tea, and is combined with the D-S evidence theory to evaluate the quality of green tea. Moreover, methods such as Pearson, RFE, PSO, and Lasso regression are utilized to evaluate six models with respect to R² value, RMSEC, RMSEP, and RPD, in order to select the optimal fusion model.

A second objective of the present disclosure is to provide application of a method for intelligent quality evaluation of instant green tea based on multi-source data fusion, where the method is used for intelligent quality identification and rapid prediction for the instant green tea.

Compared with the prior art, the present disclosure has the following advantages:
1. The present disclosure provides the method for intelligent quality evaluation of instant green tea based on multi-source data fusion and use thereof. The core lies in the organic integration of three intelligent evaluation techniques, namely computer vision, electronic nose, and electronic tongue, to construct a multi-source data fusion platform. The computer vision technology can precisely extract color features of instant green tea powder and infusion through image acquisition and processing, and these features intuitively reflect key information such as oxidation degree and pigment distribution of the instant green tea. The electronic nose technology simulates the olfactory system of mammals, enabling objective evaluation of tea aroma quality by detecting the aroma components of the instant green tea. The electronic tongue technology further simulates the human gustatory system and can accurately analyze the taste features of the instant green tea, thereby providing comprehensive data support for quality evaluation.
2. The present disclosure provides the method for intelligent quality evaluation of instant green tea based on multi-source data fusion and use thereof. Based on data acquisition, the present disclosure employs various machine learning algorithms, including SVM, RF, and KNN, to establish a variety classification model and a quality indicator prediction model for instant green tea. By training on and learning extensive experimental data, these models can accurately identify different varieties of instant green tea and predict the content of key quality indicators thereof, such as tea polyphenols, catechins, and caffeine. Experimental results demonstrate that the models based on machine learning exhibit superior performance in both classification and prediction tasks, significantly outperforming traditional chemical analysis and sensory evaluation methods.
3. The present disclosure provides the method for intelligent quality evaluation of instant green tea based on multi-source data fusion and use thereof. To further improve the accuracy and generalization capability of the models, the present disclosure further introduces feature-level and decision-level fusion strategies. The feature-level fusion strategy integrates data from different intelligent evaluation techniques to form a more comprehensive quality evaluation dataset. The decision-level fusion strategy constructs a plurality of classification or prediction models based on independent data sources and synthesizes the decision results of these models according to specific fusion rules to derive the final determination. Experiments confirm that the application of these fusion strategies significantly improves the classification accuracy and prediction precision of the models.
4. The present disclosure provides the method for intelligent quality evaluation of instant green tea based on multi-source data fusion and use thereof. The present disclosure further investigates the impact of storage time on instant green tea quality. By simulating instant green tea samples under different storage conditions and analyzing the changes in quality indicators and volatile compound content over time, a classification model for storage time of instant green tea is constructed. This model can accurately predict the storage time of instant green tea, providing a scientific basis for product shelf-life management and market sales.
5. The present disclosure provides the method for intelligent quality evaluation of instant green tea based on multi-source data fusion and use thereof. Regarding model optimization, the present disclosure introduces various feature selection methods, such as PSO, RFE, and Lasso regression. These methods improve the prediction accuracy and generalization capability of the models by optimizing the feature subsets and reducing data redundancy. Experimental results indicate that the models optimized with the feature selection methods exhibit higher accuracy and stability in both classification and prediction tasks.

In conclusion, by combining the multi-source data fusion technology with the machine learning algorithms in the present disclosure, a system and method for efficient, accurate, and comprehensive intelligent quality evaluation of instant green tea are established. This system not only enables the classification of instant green tea varieties and the prediction of the quality indicators but also provides an in-depth analysis of the impact of storage time on quality. It offers robust support for the production, quality control, and market sales of instant green tea. Moreover, by optimizing model performance with the introduced feature selection methods, the practicality and reliability of the system are further improved, paving a new path for the intelligent development of the tea industry.

### BRIEF DESCRIPTION OF THE DRAWINGS

FIGS. 1A-1B show images of a tea powder and a tea infusion captured using computer vision recognition in an embodiment of the present disclosure, where (FIG. 1A) represents the tea powder and (FIG. 1B) represents the tea infusion;

FIGS. 2A-2D show PCA results (FIG. 2A) and LDA results (FIG. 2B) of tea powders and PCA results (FIG. 2C) and LDA results (FIG. 2D) of tea infusions of different varieties of instant green tea obtained by an electronic nose in an embodiment of the present disclosure;

FIGS. 3A-3B show PCA results (FIG. 3A) and LDA results (FIG. 3B) of different varieties of instant green tea obtained by an electronic tongue in an embodiment of the present disclosure;

FIGS. 4A-4F show the contents of catechins in different varieties of instant green tea: (FIG. 4A) EGC, (FIG. 4B) C, (FIG. 4C) EC, (FIG. 4D) EGCG, (FIG. 4E) ECG, and (FIG. 4F) TC, in an embodiment of the present disclosure, where different lowercase letters indicate the presence of significant differences (p<0.05);

FIGS. 5A-5B are schematic diagrams illustrating classification and fusion strategies of a method for intelligent quality evaluation of instant green tea based on multi-source data fusion according to the present disclosure, where (FIG. 5A) depicts feature-level fusion and (FIG. 5B) depicts decision-level fusion; and

FIG. 6 is a schematic flowchart of a method for intelligent quality evaluation of instant green tea based on multi-source data fusion according to the present disclosure.

### DETAILED DESCRIPTION OF THE EMBODIMENTS

The present disclosure will be described in detail below with reference to the drawings and specific embodiments. The embodiments are implemented on the premise of the technical solutions of the present disclosure. The following presents detailed implementations and specific operation processes. The protection scope of the present disclosure, however, is not limited to the following embodiments.

In the technical solutions, features such as component models, material designations, connection structures, applications, and algorithms not explicitly specified shall be deemed as common technical features disclosed in the prior art.

In the present disclosure, unless otherwise clearly specified and defined, the terms “mount”, “interconnect”, “connect”, “fix”, and the like should be understood in a broad sense. For example, a “connection” may be a fixed connection, removable connection, or integral connection; may be a mechanical connection or electrical connection; may be a direct connection or indirect connection using a medium; and may be intercommunication or interaction between two elements. “Upper”, “lower”, “left”, “right”, and the like are used only to indicate a relative positional relationship, and when the absolute position of the described object is changed, the relative positional relationship is also changed accordingly. Those of ordinary skill in the art may understand specific meanings of the above terms in the present disclosure based on a specific situation.

It should be noted that in the present disclosure, relational terms such as first and second are merely used to distinguish one entity or operation from another entity or operation without necessarily requiring or implying any such actual relationship or order between such entities or operations. Moreover, terms “include”, “comprise” or any other variations thereof are intended to cover non-exclusive inclusions, so that a process, method, article or device including a series of elements not only includes those elements, but also includes other elements that are not explicitly listed, or also includes inherent elements of the process, method, article or device. In the case that there are no more restrictions, an element limited by the statement “includes a . . . ” does not exclude the presence of additional identical elements in the process, the method, the article, or the device that includes the element.

In the following embodiments, unless otherwise specified, all raw materials or processing techniques shall be considered conventional commercially available raw materials or conventional processing techniques in this art.

The present disclosure relates to a method for intelligent quality evaluation of instant green tea based on multi-source data fusion and use thereof and belongs to the field of food inspection, involving the establishment of a system for intelligent quality identification of instant green tea. The present disclosure mainly provides a rapid intelligent tea quality evaluation technique. The quality indicators of instant green tea are determined by chemical analysis, and relevant data is collected using an intelligent evaluation technique. Subsequently, correlation analysis is employed to reveal the relationships between the quality indicators and intelligent features. Based on independent data sources, a classification model for instant green tea varieties is constructed. By combining the multi-source data fusion technology with machine learning algorithms in the present disclosure, a system and method for efficient, accurate, and comprehensive intelligent quality evaluation of instant green tea are established. This system and method not only achieve classification of instant green tea varieties and prediction of quality indicators but also allow for an in-depth study on the impact of storage time on quality. Furthermore, by introducing feature selection methods to optimize model performance, the practicality and reliability of the system are further improved.

In the following embodiments:

A computer vision device includes a dark chamber, a bracket, a light source, a camera, and a holding container. The bracket, the light source, the camera, and the holding container are disposed inside the dark chamber. The light source and the camera are connected to the bracket. The camera, the light source, and the holding container are arranged sequentially from top to bottom. The holding container is configured to hold the tea infusion or the tea powder. The dark chamber includes a black matte acrylic plate and is in a cubic shape. A side sliding groove and a bottom groove are formed in a front side surface of a body of the dark chamber to allow a front panel to slide up and down and be inserted into the bottom groove, thereby forming an enclosed space inside the dark chamber to inhibit an external light environment from affecting sample collection. The bracket includes a carrying platform, a support rod, and an adjusting bracket. The carrying platform is connected to the support rod, and the adjusting bracket is connected to the support rod. A stepped circular groove is designed at a central point of the carrying platform to secure the holding container to ensure a consistent position for each collection. The adjusting bracket is configured to adjust distances from the light source and the camera to the container. The light source is a ring-shaped shadowless light source. The camera is connected to a computer via a data cable, and parameters of the camera are adjusted using software to achieve image acquisition.

The dark chamber includes a black matte acrylic plate and is in a cubic shape (400 mm×400 mm×600 mm); and a side sliding groove and a bottom groove are formed in a front side surface of a body of the dark chamber to allow a front panel to slide up and down and be inserted into the bottom groove, thereby forming an enclosed space inside the dark chamber to inhibit an external light environment from affecting sample collection;

The bracket includes a carrying platform (300 mm×300 mm), a support rod (400 mm high), and an adjusting bracket. The carrying platform is connected to the support rod, and the adjusting bracket is connected to the support rod. The stepped circular groove is designed at the central point of the carrying platform to secure the holding container to ensure the consistent position for each collection. The adjusting bracket is configured to adjust distances from the light source and the camera to the container.

The light source is a D65 ring-shaped shadowless light source, as specified by the International Commission on Illumination (CIE), which approximates the true resolution of daylight and has a color temperature of 6500 K, an inner diameter of 75 mm, and an outer diameter of 120 mm. The light source can emit uniform and consistent light, thereby enabling the acquisition of higher-quality digital image information.

The camera, selected as Nikon D5100 (CMOS), is connected to a computer via a data cable, and parameters of the camera are adjusted using software to achieve image acquisition.

The electronic nose is the CNose-14 electronic nose manufactured by Shanghai BosinTech Industrial Development Co., Ltd, which is composed of a sampling system, a sensor array, a signal acquisition system, and a gas purging channel. The sampling system is responsible for collecting a sample gas and controlling a gas flow rate. The sensor array is the core component of the electronic nose system for detecting the sample gas and is composed of 14 imported metal oxide semiconductor (MOS) sensors with different properties, each exhibiting varying sensitivities to different volatile compounds. The signal acquisition system can control the acquisition of response signals by the electronic nose system.

For flavor acquisition by the electronic tongue, the Smart Tongue electronic tongue manufactured by Shanghai BosinTech Industrial Development Co., Ltd. is used, which is composed of a sampling system, a sensor array, and a data acquisition and analysis system. The sensor array is composed of 6 cross-sensitive chemical sensors (platinum S1, gold S2, palladium S3, tungsten S4, titanium S5, and silver S6). Before each test, the electronic tongue system needs to be pre-heated for more than 30 minutes. The sensors are cleaned with deionized water, and it is confirmed that the protective solution in the Ag/AgCl reference electrode is not dry. During sample detection, the sample to be tested in the beaker is immersed to cover the bottom of the electrode. This allows the taste signals to cause potential changes across the lipid-sensitive membrane via the sensor array, which are then converted into electrical signals. Finally, software is used to receive, record, and process electrical signal response values, and save them on the computer.

### Embodiment

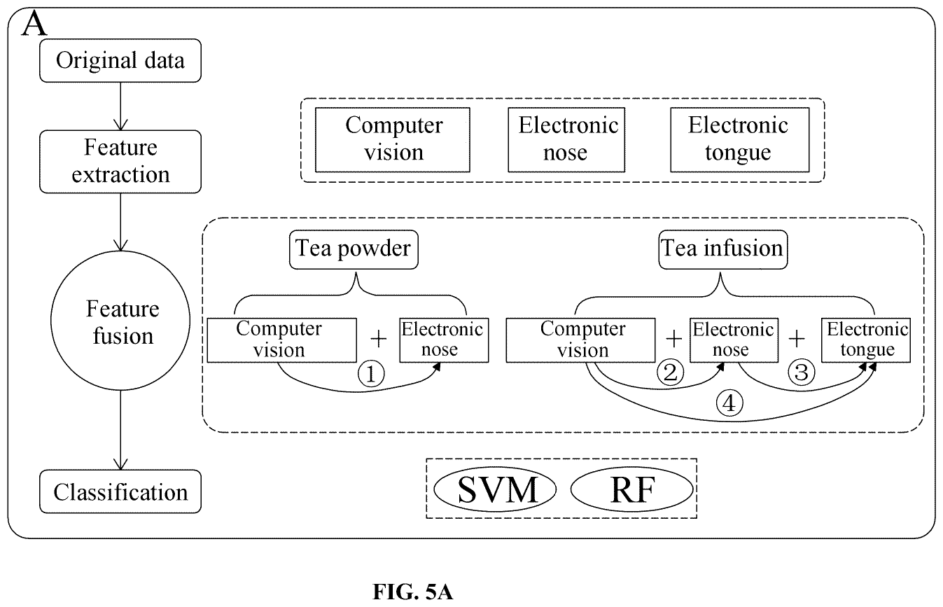

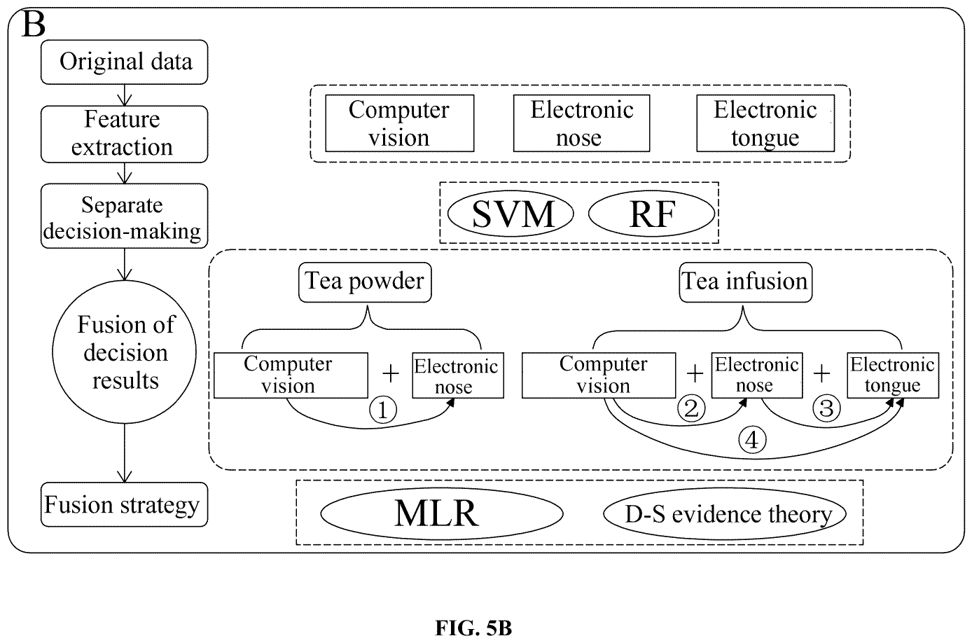

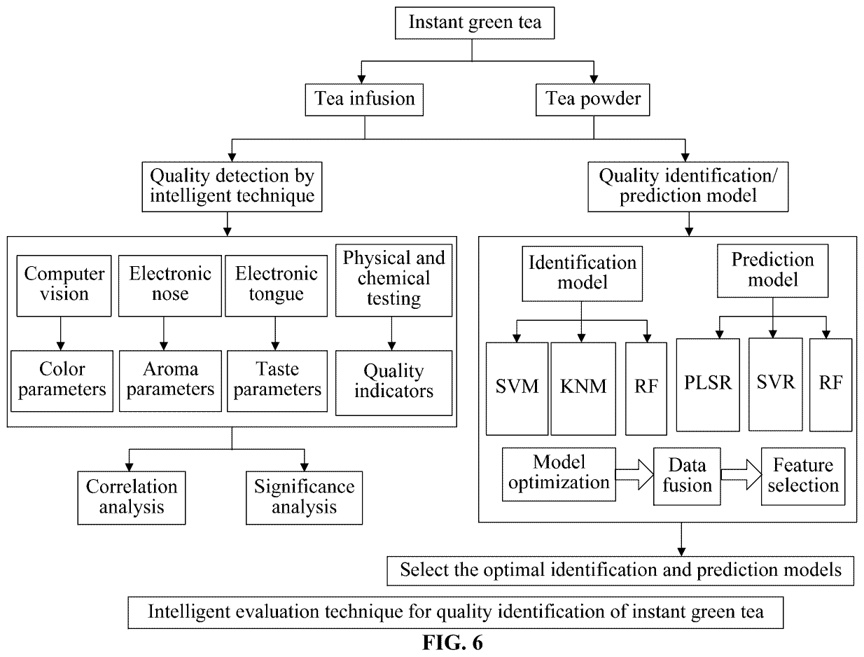

As shown in FIGS. 5A-5B and FIG. 6, this embodiment provides a method for intelligent quality evaluation of instant green tea based on multi-source data fusion and use thereof. The method includes:
step 1, selecting instant green tea as a raw material; brewing one portion of the instant green tea with boiling water to form a tea infusion, while keeping the other portion of the instant green tea as a tea powder; using a computer vision device to collect visual images of the tea infusion and the tea powder; using an electronic nose to acquire aroma information of the tea infusion and the tea powder; using an electronic tongue to collect taste information of the tea infusion; and applying high-performance liquid chromatography to measure information of quality components in the tea infusion and the tea powder;
step 2, performing dimensionality reduction separately on the aroma information, the visual images, and the taste information that are used as data of feature parameters; after the dimensionality reduction, dividing a dataset; determining quality indicators of the tea infusion and the tea powder; and performing correlation analysis on the quality indicators and the feature parameters to form a fused feature set;
step 3, selecting features from the fused feature set formed in step 2 to form an optimal feature subset; constructing, based on the optimal feature subset, regression and classification models of SVM, KNN, and RF, and prediction models of PLSR, SVR, and RFR to predict contents of the quality indicators of the instant green tea, and using indicators of classification accuracy rate, R² value of training and prediction sets, RMSEC, RMSEP, and RPD as evaluation criteria; and
step 4, applying data fusion at a decision level and a feature level based on the optimal feature subset formed in step 3; applying an MLR model and D-S evidence theory in a model architecture at the decision level and introducing a feature selection method at the feature level to perform multi-source fusion on the data separately; deriving fusion models based on evaluation results to improve accuracy and stability of classification and prediction; for variety identification of the instant green tea, applying the model architecture at the decision level; for quality identification of the instant green tea, applying the model architecture at the feature level; and using the R² value, the RMSEC, the RMSEP, and the RPD as evaluation indicators for evaluation so as to select an optimal fusion model. The models applied in step 4 are primarily based on statistical methods for feature fusion, which are categorized into distinct fusion strategies such as decision-level fusion and feature-level fusion. In contrast, the models used in step 3 are regression and classification and prediction models. This reflects a progressive relationship that follows a sequence of prediction first, followed by feature correlation analysis.

The specific steps are as follows.

Six varieties of instant green tea (purchased from Zhejiang Mingbao Company): G302, G305, G306, G307, J753, and ZTHG505, are selected as samples in both tea powder and tea infusion forms for detection by computer vision, the electronic nose, and the electronic tongue.

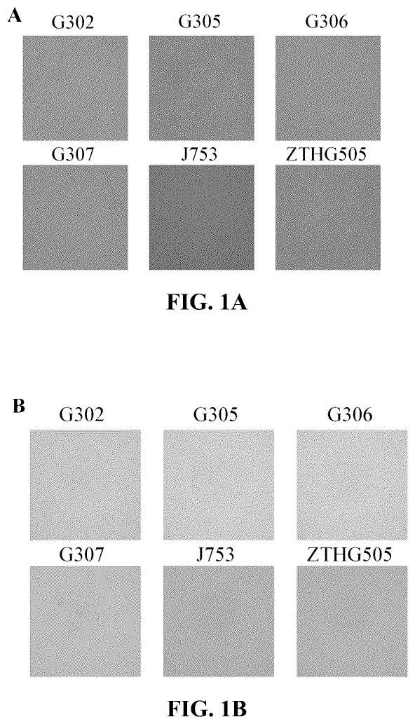

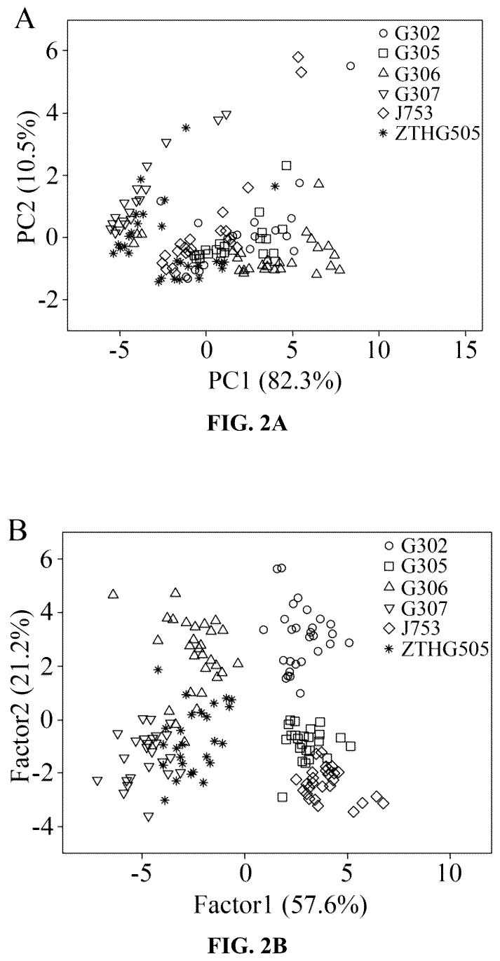

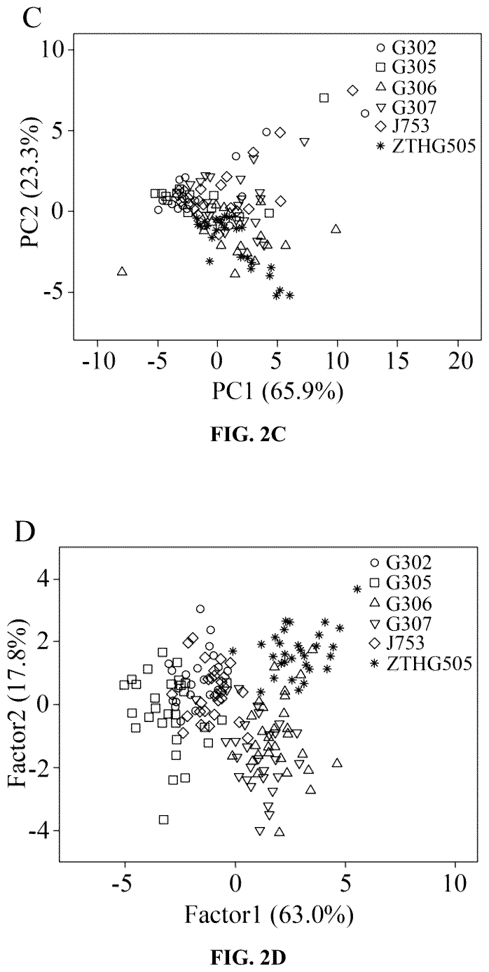

In computer vision detection, a self-assembled computer vision device is employed to capture images of tea powder and tea infusion samples. 3 g of tea powder sample is evenly spread in a glass dish. The tea infusion sample is prepared by steeping 0.5 g of tea powder in 150 mL of ultrapure boiling water, and then an equal amount of the tea infusion sample is transferred into a porcelain tasting cup. All images are acquired under identical conditions and saved in JPG format. Subsequently, Python software is employed for image preprocessing, automatically selecting a region of interest of 600×600 pixels near the center, and extracting a plurality of color feature parameters such as red, green, and blue channels as well as hue, saturation, and lightness. Each sample is prepared six times, with five replicate measurements per preparation, yielding a total of 360 sets of data. The images of the tea powder and the tea infusion obtained using computer vision recognition are specifically shown in FIGS. 1A-1B, where FIG. 1A corresponds to the tea powder and FIG. 1B corresponds to the tea infusion. Before detection by the electronic nose, preliminary experiments are conducted to determine optimal detection parameters. For tea powder samples, 5 g is used with a headspace time of 60 min at an ambient temperature of 25° C.; and for tea infusion samples, 15 mL is used under otherwise identical conditions. After sealing and resting in headspace vials, sample gases are drawn at a specific rate for detection. Each sample is likewise repeated six times with five replicate detections per repetition, resulting in a total of 360 sets of data. As shown in FIGS. 2A-2D, the results of different varieties of instant green tea obtained by the electronic nose are presented schematically, where FIG. 2A shows the PCA results for the tea powder, FIG. 2B shows the LDA results for the tea powder, FIG. 2C shows the PCA results for the tea infusion, and FIG. 2D shows the LDA results for the tea infusion.

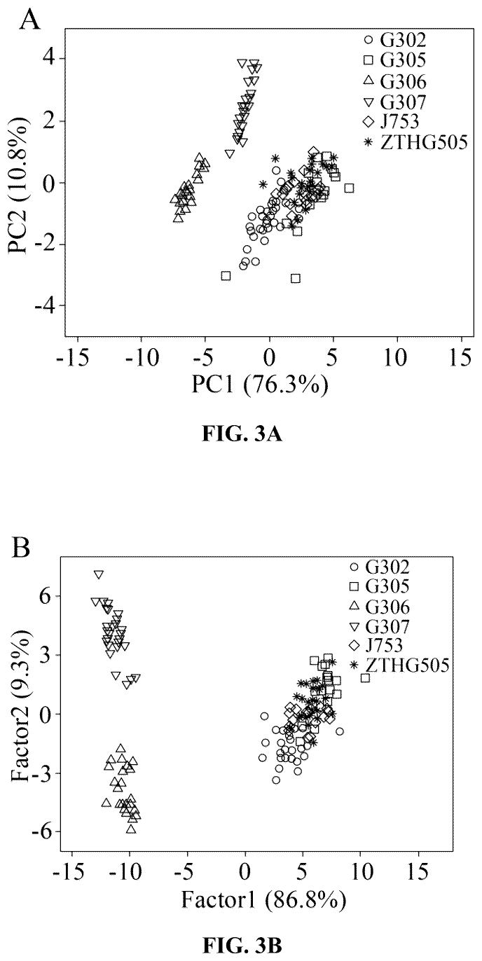

For detection by the electronic tongue, the optimal parameters for the tea infusion samples are determined based on the preliminary experiments. 0.5 g of tea powder is dissolved in 150 mL of ultrapure water, and 30 mL of the infusion is taken as the sample to be tested. After setting the acquisition frequency, the sensor signal amplification factor, and the detection time, the sample is immersed to cover the electrode bottom for detection, and the data is exported for analysis. Each sample is repeated six times with five replicate detections per repetition, resulting in a total of 180 sets of data. As shown in FIGS. 3A-3B, the results of different varieties of instant green tea obtained by the electronic tongue are presented schematically, where FIG. 3A shows the PCA results for the tea infusion and FIG. 3B shows the LDA results for the tea infusion.

The determination of the quality indicators of the tea infusion and the tea powder includes the following process:

The components in tea such as TPs, FAAs, the TP/FAA ratio, CAF, EGC, C, EC, and EGCG are determined by liquid chromatography; the tea sample is ground into powder, homogenized, and then subjected to ultrasonic extraction with ultrapure water or a specific solvent; the extract is centrifuged; and the supernatant is filtered for subsequent detection. A C-18 reversed-phase chromatography column is selected, and the contents of TPs, FAAs, CAF, and C, are measured according to national standards, with the corresponding data recorded.

Pearson correlation analysis is performed on 9 color feature parameters and the quality indicators of the instant green tea infusion and the tea powder. Meanwhile, the electronic nose technology is employed to extract 14 aroma feature parameters from the tea infusion and the tea powder, followed by an in-depth analysis of the correlations between these aroma features and 10 quality indicators. Additionally, correlation analysis is also performed on 18 taste feature parameters extracted by an electronic tongue sensor and the 10 quality indicators of the instant green tea. The TP content is measured according to GB/T 8313-2018 “Determination of total polyphenols and catechins content in tea”. The contents of catechins and CAF are measured according to GB/T 8313-2018 “Determination of total polyphenols and catechins content in tea”. The FAA content is measured according to GB/T 8314-2013 “Determination of free amino acids content in tea”.

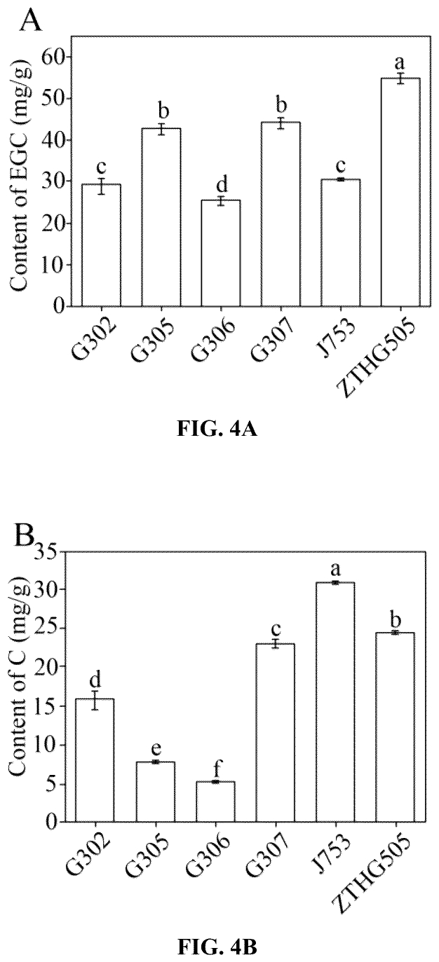

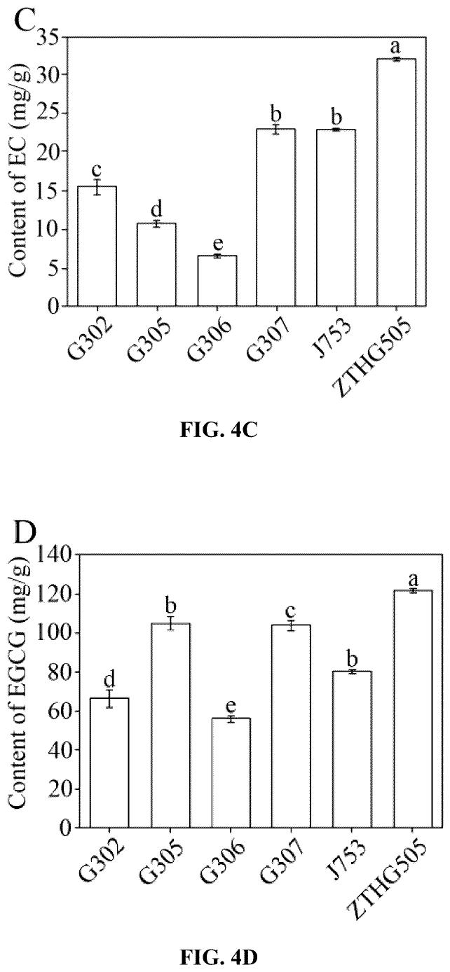

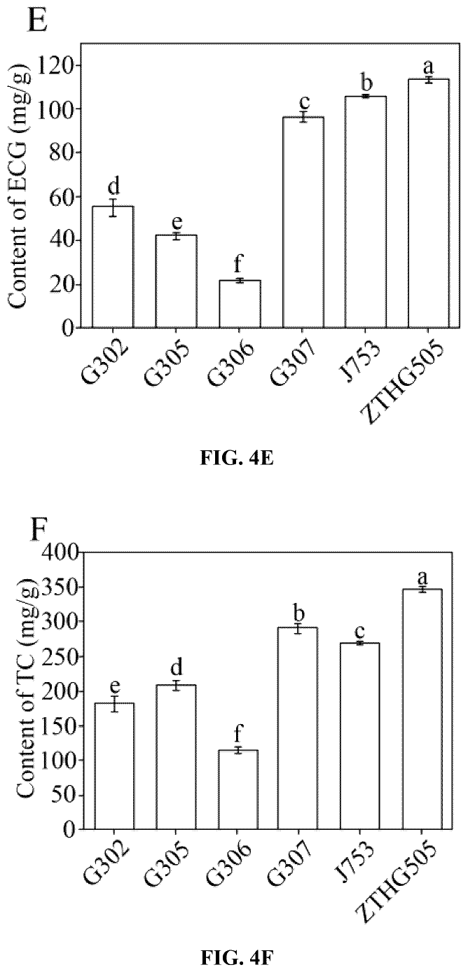

During the modeling process, two approaches are adopted: classification models and regression models. The classification models (or regression and classification models) include SVM, KNN, and RF, which are used for qualitative discrimination of instant green tea varieties. The prediction models include PLSR, SVR, and RFR, which are employed for rapid quantitative prediction of the quality indicators. As shown in FIGS. 4A-4F, the contents of catechins such as EGC, C, EC, EGCG, ECG, and TC in different varieties of instant green tea are presented schematically.

Model evaluation indicators in the evaluation criteria and calculation methods thereof are as follows:

$$classification accuracy rate A = \frac{{N}_{c}}{N} ;$$

$$determination coefficient {R}^{2} value {R}^{2} = \frac{( {\sum }_{i = 1}^{n} {X}_{i} - \overline{X} ) {( {Y}_{i} - \overline{Y} )}^{2}}{{\sum }_{i = 1}^{n} ( {X}_{i} - \overline{X} ) {\sum }_{i = 1}^{n} ( {Y}_{i} - \overline{Y} )} ;$$

$$root mean square error of calibration R M S E C = \sqrt{\frac{{\sum }_{i = 1}^{n} {( {X}_{ci} - {Y}_{ci} )}^{2}}{n}} ;$$

$$root mean square error of prediction R M S E P = \sqrt{\frac{{\sum }_{i = 1}^{n} {( {X}_{pi} - {Y}_{pi} )}^{2}}{n}} ;$$

$$standard deviation S D = \sqrt{\frac{{\sum }_{i = 1}^{n} {( {X}_{i} - \overline{X} )}^{2}}{n - 1}} ; and$$

$$relative percentage deviation R P D = \frac{S D}{R M S E P} .$$

The dataset is subjected to z-score standardization prior to modeling to eliminate the dimensional influence. The data is divided into a 70% training set and a 30% test set, and model performance is evaluated using 10-fold cross-validation. Specifically, the original dataset is evenly split into 10 mutually exclusive subsets. 9 subsets are used for training and the remaining 1 subset for testing each time. This process is repeated 10 times, and the average result is taken as the final evaluation result, thereby improving the reliability and generalization capability of evaluation.

For the classification models, the classification accuracy rate is used for performance evaluation. For the prediction models, the R², the RMSEC, the RMSEP, and the RPD serve as the evaluation indicators. Higher values of the R² and the RPD, along with lower values of the RMSEC and the RMSEP, indicate greater model reliability. When the RPD is greater than or equal to 3.0, the predictive performance of the model is significant; when the RPD is between 2.5 and 3.0, the prediction is considered acceptable; and when the RPD is less than or equal to 2.5, further optimization is required. The primary model evaluation indicators are as described above to ensure comprehensiveness and accuracy in model evaluation.

The classification fusion strategy involves comprehensive analysis of data extracted by the computer vision, the electronic nose, and the electronic tongue through feature-level fusion and decision-level fusion to construct the classification models. In the feature-level fusion strategy, color features (9-dimensional), aroma features (14-dimensional), and taste features (18-dimensional) representing the tea powder and the tea infusion of the instant green tea are first extracted from the raw information of the computer vision, the electronic nose, and the electronic tongue. Subsequently, the features of the tea powder and the tea infusion are pairwise merged to generate four new dual-source fused feature sets. Finally, SVM and RF classification models are constructed based on the fused feature datasets. Following the same feature extraction process as in the feature-level fusion strategy, corresponding SVM and RF classification models are independently established based on the features of each data source to obtain respective decision results. By integrating the model decision results from the computer vision, the electronic nose, and the electronic tongue, these are then fed into the MLR model and the D-S evidence theory to achieve decision-level data fusion, ultimately generating the classification results.

The results of multi-feature fusion are as shown in the following Tables 1 to 4.

TABLE 1
Classification Results of Varieties of Instant Green Tea
by SVM and RF based on Feature-Level Fusion Strategy

| | | | | 10-Fold Cross- | |
|---|---|---|---|---|---|
| Fused Feature | | Parameter | | Validation Set | Prediction Set |
| Dataset | Classifier | C | gamma/N | Accuracy Rate | Accuracy Rate |
| Computer vision tea | SVM | 27.5934 | 0.01 | 100% | 98.15% |
| powder + electronic | RF | / | 80 | 98.40% | 98.15% |
| nose tea powder | | | | | |
| Computer vision tea | SVM | 100 | 0.01 | 97.69% | 92.59% |
| infusion + electronic | RF | / | 200 | 98.46% | 94.44% |
| nose tea infusion | | | | | |
| Computer vision tea | SVM | 100 | 0.01 | 97.56% | 98.15% |
| infusion + electronic | RF | / | 140 | 98.40% | 100% |
| tongue | | | | | |
| Electronic nose tea | SVM | 6.9059 | 0.01 | 91.15% | 87.04% |
| infusion + electronic | RF | / | 200 | 92.82% | 94.44% |
| tongue | | | | | |

TABLE 2
Classification Results of MLR Model Based on Decision-level Fusion Strategy

| | | | | | | | | 10-Fold | |
|---|---|---|---|---|---|---|---|---|---|
| | | | | | | | | Cross- | |
| | | | | | | | | Validation | Prediction |
| Decision | | | | | | | | Set | Set |
| Fusion | Decision | Classification Accuracy Rate of Each Variety of Instant Green Tea | | | | | | Accuracy | Accuracy |
| Model | Classifier | G302 | G305 | G306 | G307 | J753 | ZTHG505 | Rate | Rate |
| Computer | SVM | 100% | 88.89% | 42.86% | 100% | 100% | 88.89% | 97.56% | 88.89% |
| vision tea | RF | 100% | 66.67% | 100% | 80.00% | 100% | 77.78% | 100.00% | 87.04% |
| powder + | | | | | | | | | |
| electronic | | | | | | | | | |
| nose tea | | | | | | | | | |
| powder_MLR | | | | | | | | | |
| Computer | SVM | 77.78% | 77.78% | 100% | 90.00% | 80.00% | 77.78% | 92.05% | 83.33% |
| vision tea | RF | 44.44% | 77.78% | 100% | 80.00% | 90.00% | 88.89% | 100.00% | 79.63% |
| infusion + | | | | | | | | | |
| electronic | | | | | | | | | |
| nose tea | | | | | | | | | |
| infusion_MLR | | | | | | | | | |
| Computer | SVM | 77.78% | 100% | 100% | 90.00% | 70.00% | 88.89% | 97.56% | 87.04% |
| vision tea | RF | 100% | 55.56% | 100% | 90.00% | 60.00% | 77.78% | 100.00% | 79.63% |
| infusion + | | | | | | | | | |
| electronic | | | | | | | | | |
| tongue_MLR | | | | | | | | | |
| Electronic | SVM | 88.89% | 44.44% | 85.71% | 90.00% | 60.00% | 77.78% | 87.31% | 74.07% |
| nose tea | RF | 44.44% | 66.67% | 100% | 90.00% | 60.00% | 66.67% | 100.00% | 70.37% |
| infusion + | | | | | | | | | |
| electronic | | | | | | | | | |
| tongue_MLR | | | | | | | | | |

TABLE 3
Classification Results of D-S Evidence Theory Based on Decision-level Fusion Strategy

| | | | | | | | | 10-Fold | |
|---|---|---|---|---|---|---|---|---|---|
| | | | | | | | | Cross- | |
| | | | | | | | | Validation | Prediction |
| Decision | | | | | | | | Set | Set |
| Fusion | Decision | Classification Accuracy Rate of Each Variety of Instant Green Tea | | | | | | Accuracy | Accuracy |
| Model | Classifier | G302 | G305 | G306 | G307 | J753 | ZTHG505 | Rate | Rate |
| Computer | SVM | 100% | 66.67% | 100% | 100% | 100% | 100% | 100% | 94.44% |
| vision tea | RF | 100% | 100% | 85.71% | 100% | 100% | 100% | 100% | 98.15% |
| powder + | | | | | | | | | |
| electronic | | | | | | | | | |
| nose tea | | | | | | | | | |
| powder_DS | | | | | | | | | |
| Computer | SVM | 77.78% | 100% | 100% | 90.00% | 100% | 100% | 100% | 94.44% |
| vision tea | RF | 100% | 100% | 100% | 100% | 100% | 100% | 100% | 100% |
| infusion + | | | | | | | | | |
| electronic | | | | | | | | | |
| nose tea | | | | | | | | | |
| infusion_DS | | | | | | | | | |
| Computer | SVM | 100% | 100% | 100% | 100% | 90.00% | 100% | 100% | 98.15% |
| vision tea | RF | 100% | 100% | 100% | 100% | 100% | 100% | 100% | 100% |
| infusion + | | | | | | | | | |
| electronic | | | | | | | | | |
| tongue_DS | | | | | | | | | |
| Electronic | SVM | 100% | 77.78% | 100% | 100% | 80.00% | 100% | 100% | 92.59% |
| nose tea | RF | 100% | 88.89% | 100% | 100% | 100% | 100% | 100% | 96.30% |
| infusion + | | | | | | | | | |
| electronic | | | | | | | | | |
| tongue_DS | | | | | | | | | |

TABLE 4
Prediction Results of Contents of Quality Indicators Based on
Fused Feature Set

| Quality | | Training Set | | Prediction Set | | |
|---|---|---|---|---|---|---|
| Indicator | N | R² | RMSEC | R² | RMSEP | RPD |
| TP | 80 | 0.9753 | 7.3606 | 0.9689 | 10.8127 | 5.6883 |
| FAA | 120 | 0.9505 | 1.2710 | 0.9359 | 1.9482 | 4.1111 |
| TP/FAA | 120 | 0.9937 | 0.0514 | 0.9921 | 0.0589 | 11.2888 |
| CAF | 60 | 0.9801 | 1.3768 | 0.9707 | 1.9932 | 5.8642 |
| EGC | 300 | 0.9290 | 2.5714 | 0.9569 | 1.9306 | 4.8368 |
| C | 40 | 0.9794 | 0.6815 | 0.9773 | 1.4202 | 6.6723 |
| EC | 20 | 0.9914 | 0.4886 | 0.9566 | 1.6137 | 4.8122 |
| EGCG | 20 | 0.9252 | 5.3390 | 0.9077 | 6.5367 | 3.3094 |
| ECG | 80 | 0.9983 | 1.2006 | 0.9539 | 8.5242 | 3.9511 |

As can be seen from Tables 1 to 4, this embodiment achieves the organic integration of three intelligent evaluation techniques, namely computer vision, electronic nose, and electronic tongue, to construct a multi-source data fusion platform. The computer vision technology can precisely extract color features of instant green tea powder and infusion through image acquisition and processing, and these features intuitively reflect key information such as oxidation degree and pigment distribution of the instant green tea. The electronic nose technology simulates the olfactory system of mammals, enabling objective evaluation of tea aroma quality by detecting the aroma components of the instant green tea. The electronic tongue technology further simulates the human gustatory system and can accurately analyze the taste features of the instant green tea, thereby providing comprehensive data support for quality evaluation.

The above embodiments are only used for describing the technical solutions of the present disclosure, and are not intended to limit the present disclosure. It will be appreciated by those skilled in the art that, although the present disclosure is described in detail with reference to the foregoing embodiments, modification can still be made to the technical solutions in the foregoing embodiments, or equivalent replacements can be made to part of technical features therein. These modifications or replacements do not go beyond the protection scope of the present disclosure which is defined by the appended claims.

What is claimed is:

1. A method for intelligent quality evaluation of instant green tea based on multi-source data fusion, comprising:
step 1, selecting instant green tea as a raw material; brewing one portion of the instant green tea with boiling water to form a tea infusion, while keeping another portion of the instant green tea as a tea powder; using a computer vision device to collect visual images of the tea infusion and the tea powder; using an electronic nose to acquire aroma information of the tea infusion and the tea powder; using an electronic tongue to collect taste information of the tea infusion; and applying high-performance liquid chromatography to measure information of quality components in the tea infusion and the tea powder;
step 2, performing dimensionality reduction separately on the aroma information, the visual images, and the taste information that are used as data of feature parameters; after the dimensionality reduction, dividing a dataset; determining quality indicators of the tea infusion and the tea powder; and performing correlation analysis on the quality indicators and the feature parameters to form a fused feature set;
step 3, selecting features from the fused feature set formed in step 2 to form an optimal feature subset; constructing, based on the optimal feature subset, regression and classification models and a prediction model to predict contents of the quality indicators of the instant green tea, and using indicators of classification accuracy rate, R² value of training and prediction sets, root mean square error of calibration (RMSEC), root mean square error of prediction (RMSEP), and relative prediction deviation (RPD) as evaluation criteria; and
step 4, applying data fusion at a decision level and a feature level based on the optimal feature subset formed in step 3; applying a multiple linear regression model and Dempster-Shafer (D-S) evidence theory in a model architecture at the decision level and introducing a feature selection method in a model architecture at the feature level to perform multi-source fusion on the data separately; deriving fusion models based on evaluation results; for variety identification of the instant green tea, applying the model architecture at the decision level; for quality prediction of the instant green tea, applying the model architecture at the feature level; and using the R² value, the RMSEC, the RMSEP, and the RPD as evaluation indicators for evaluation so as to select an optimal fusion model.

2. The method for intelligent quality evaluation of instant green tea based on multi-source data fusion according to claim 1, wherein the computer vision device comprises:
a dark chamber, a bracket, a light source, a camera, and a holding container;
wherein the bracket, the light source, the camera, and the holding container are disposed inside the dark chamber;
wherein the light source and the camera are connected to the bracket;
wherein the camera, the light source, and the holding container are arranged sequentially from top to bottom; and
wherein the holding container is configured to hold the tea infusion or the tea powder.

3. The method for intelligent quality evaluation of instant green tea based on multi-source data fusion according to claim 2, wherein the dark chamber comprises a black matte acrylic plate and is in a cubic shape; and a side sliding groove and a bottom groove are formed in a front side surface of a body of the dark chamber to allow a front panel to slide up and down and be inserted into the bottom groove, thereby forming an enclosed space inside the dark chamber to inhibit an external light environment from affecting sample collection;
wherein the bracket comprises a carrying platform, a support rod, and an adjusting bracket; the carrying platform is connected to the support rod, and the adjusting bracket is connected to the support rod; a stepped circular groove is disposed and configured at a central point of the carrying platform to secure the holding container to ensure a consistent position for each collection; and the adjusting bracket is configured to adjust distances from the light source and the camera to the container; and
wherein the light source is a ring-shaped shadowless light source;
wherein the camera is connected to a computer via a data cable, and parameters of the camera are adjustable using software to achieve image acquisition.

4. The method for intelligent quality evaluation of instant green tea based on multi-source data fusion according to claim 1, wherein an olfactory sensing detection system comprises the electronic nose; and
wherein a gustatory sensing system comprises the electronic tongue.

5. The method for intelligent quality evaluation of instant green tea based on multi-source data fusion according to claim 1, wherein the regression and classification models comprise one or more of support vector machine (SVM), K-nearest neighbor (KNN), and random forest (RF); and
the prediction model comprises one or more of partial least squares regression (PLSR), support vector regression (SVR), and RF.

6. The method for intelligent quality evaluation of instant green tea based on multi-source data fusion according to claim 1, wherein model evaluation indicators in the evaluation criteria and calculation methods are as follows:
$$classification accuracy rate A = \frac{{N}_{c}}{N} ;$$
$$determination coefficient {R}^{2} value {R}^{2} = \frac{( {\sum }_{i = 1}^{n} {X}_{i} - \overline{X} ) {( {Y}_{i} - \overline{Y} )}^{2}}{{\sum }_{i = 1}^{n} ( {X}_{i} - \overline{X} ) {\sum }_{i = 1}^{n} ( {Y}_{i} - \overline{Y} )} ;$$
$$root mean square error of calibration R M S E C = \sqrt{\frac{{\sum }_{i = 1}^{n} {( {X}_{ci} - {Y}_{ci} )}^{2}}{n}} ;$$
$$root mean square error of prediction R M S E P = \sqrt{\frac{{\sum }_{i = 1}^{n} {( {X}_{pi} - {Y}_{pi} )}^{2}}{n}} ;$$
$$standard deviation S D = \sqrt{\frac{{\sum }_{i = 1}^{n} {( {X}_{i} - \overline{X} )}^{2}}{n - 1}} ; and$$
$$relative percentage deviation R P D = \frac{S D}{R M S E P} ,$$
wherein n represents a number of samples in a dataset; Xᵢ represents an actual value of an i-th sample during prediction model construction; X̅ represents a mean of actual values of all samples during the prediction model construction; Yᵢ represents a predicted value of the i-th sample during the prediction model construction; and Y̅ represents a mean of predicted values of all samples during the prediction model construction.

7. The method for intelligent quality evaluation of instant green tea based on multi-source data fusion according to claim 1, wherein the determining quality indicators of the tea infusion and the tea powder comprises:
determining tea polyphenols, total free amino acids, a ratio of tea polyphenols to total free amino acids, caffeine, epigallocatechin, catechin, epicatechin, and epigallocatechin gallate using liquid chromatography.

8. The method for intelligent quality evaluation of instant green tea based on multi-source data fusion according to claim 1, wherein in step 2, the dimensionality reduction is dimensionality reduction with principal component analysis (PCA).

9. The method for intelligent quality evaluation of instant green tea based on multi-source data fusion according to claim 1, wherein the feature selection method comprises one or more of four feature selection methods: Pearson score, recursive feature elimination, particle swarm optimization, and Lasso regression.

10. Use of the method for intelligent quality evaluation of instant green tea based on multi-source data fusion according to claim 1, wherein the method is used for intelligent quality identification and prediction of the instant green tea.

11. Use of the method for intelligent quality evaluation of instant green tea based on multi-source data fusion according to claim 2, wherein the method is used for intelligent quality identification and prediction of the instant green tea.

12. Use of the method for intelligent quality evaluation of instant green tea based on multi-source data fusion according to claim 3, wherein the method is used for intelligent quality identification and prediction of the instant green tea.

13. Use of the method for intelligent quality evaluation of instant green tea based on multi-source data fusion according to claim 4, wherein the method is used for intelligent quality identification and prediction of the instant green tea.

14. Use of the method for intelligent quality evaluation of instant green tea based on multi-source data fusion according to claim 5, wherein the method is used for intelligent quality identification and prediction of the instant green tea.

15. Use of the method for intelligent quality evaluation of instant green tea based on multi-source data fusion according to claim 6, wherein the method is used for intelligent quality identification and prediction of the instant green tea.

16. Use of the method for intelligent quality evaluation of instant green tea based on multi-source data fusion according to claim 7, wherein the method is used for intelligent quality identification and prediction of the instant green tea.

17. Use of the method for intelligent quality evaluation of instant green tea based on multi-source data fusion according to claim 8, wherein the method is used for intelligent quality identification and prediction of the instant green tea.

18. Use of the method for intelligent quality evaluation of instant green tea based on multi-source data fusion according to claim 9, wherein the method is used for intelligent quality identification and prediction of the instant green tea.
